(19) United States
(12) Patent Application Publication
(10) Pub. No.: US 2025/0180423 A1
(43) Pub. Date: Jun. 5, 2025

(54) PIEZORESISTIVE SENSOR ELEMENT AND PIEZORESISTIVE PRESSURE SENSOR WITH MINIMIZED LONG-TERM DRIFT

(71) Applicants: TE Connectivity Solutions GmbH, Schaffhausen (CH); First Sensor AG, Berlin (DE)

(72) Inventors: Kaspar JENNI, Bevaix (CH); Claire JEAN-MISTRAL, Bevaix (CH); Rafael TEIPEN, Berlin (DE); Kai NÖRTHEMANN, Berlin (DE); Alireza NIKPOURIAN, Berlin (DE)

(21) Appl. No.: 18/965,068

(22) Filed: Dec. 2, 2024

(30) Foreign Application Priority Data
Dec. 4, 2023 (EP) 23213911.3

(51) Int. Cl. G01L 9/00

(52) U.S. Cl. CPC G01L 9/0052

(57) ABSTRACT

A piezoresistive sensor element is provided that detects mechanical stress acting on a membrane. The piezoresistive sensor element includes a substrate, which in operation is subjected to mechanical stress in response to a measurand to be measured, a first array of at least four sensitive piezoresistors, wherein the sensitive piezoresistors are arranged on the substrate and are connected to form a first Wheatstone bridge for generating a first bridge signal, a second array of at least four insensitive piezoresistors, wherein the insensitive piezoresistors are connected to form a second Wheatstone bridge for generating a second bridge signal, wherein the sensitive piezoresistors have a stress sensitivity which is higher than the stress sensitivity of the insensitive piezoresistors, and wherein an output signal of the piezoresistive sensor element is generated based on a difference of the first and second bridge signals.

### CROSS REFERENCE TO RELATED APPLICATIONS

This application claims the benefit of EP Application Serial No. 23213911.3, filed 4 Dec. 2023, the subject matter of which is herein incorporated by reference in its entirety.

### BACKGROUND OF THE INVENTION

The subject matter herein relates to piezoresistive sensors. In particular, the subject matter herein relates to a piezoresistive sensor element and to a piezoresistive pressure sensor that detects mechanical stress acting on a membrane.

The piezoresistive sensor element according to the present disclosure is based on the physical principle of piezoresistivity, which will be outlined shortly in the following.

Piezoresistive sensors are among the first Micro-Electro-Mechanical-Systems (MEMS) devices and comprise a substantial market share of MEMS sensors in the market today. In particular, silicon piezoresistance has been widely used for various sensors including pressure sensors, accelerometers, cantilever force sensors, inertial sensors, and strain gauges. A detailed overview is for instance given in Barlian, A. Alvin & Park, Woo-Tae ε Mallon, Joseph R., Jr. ε Rastegar, Ali J. ε Pruitt, Beth L. (2009): “Review: Semiconductor Piezoresistance for Microsystems”, Proceedings of the IEEE. Institute of Electrical and Electronics Engineers, 97, pp. 513-552, DOI: 10.1109/JPROC.2009.2013612.

It is known that the electrical resistance (R) of a homogeneous material is a function of its dimensions and resistivity (p),

$$R = \frac{ρ \cdot l}{a} \qquad ( 1 )$$

where 1 is length, and a is average cross-sectional area.

The change in resistance due to applied stress is a function of geometry and resistivity changes. The cross-sectional area of a bulk material reduces in proportion to the longitudinal strain by its Poisson's ratio, v, which for most metals ranges from 0.20 to 0.35. For anisotropic silicon, the effective directional Poisson's ratio ranges from 0.06 to 0.36. The isotropic lower and upper limit for v are −1.0 and 0.5. The so-called gauge factor (GF) of a strain gauge is defined as

$$GF = \frac{Δ R / R}{ε} \qquad ( 2 )$$

where ε is strain and ΔR/R is fractional resistance change with strain. The change in resistance is due to both the geometric effects (1+2 v) and the fractional change in resistivity (Δρ/ρ) of the material with strain

$$\frac{Δ R}{R} = ( 1 + 2 v ) ε + \frac{Δρ}{ϱ} \qquad ( 3 )$$

Geometric effects alone provide a GF of approximately 1.4 to 2.0, and the change in resistivity, Δρ/p, for a metal is small—on the order of 0.3. However, for silicon and germanium in certain directions, Δρ/ρ is 50-100 times larger than the geometric term. For a semiconductor, elasticity and piezoresistivity are direction-dependent under specified directions of loads (stress, strain) and fields (potentials, currents). For the sensors according to the present disclosure, the stress induced change of resistivity (the so-called piezoresistive effect) is responsible for the generation of the electrical output signal.

In the following, some basics about the notation and fundamentals of piezoresistivity in semiconductors will be discussed.

As this is generally known, Miller indices can be used for describing crystal structure. Crystals have periodic arrangements of atoms arranged in one of 14 lattice types and complete reviews are available elsewhere. The Miller indices specify crystal planes by n-tuples. A direction (also referred to as a crystal direction) index [hk1] denotes a vector normal to a plane (also referred to as a crystal plane) described by (hk1), and t represents a family of planes equivalent to (hk1) by symmetry. Angle-bracketed indices, like (hk1), represent all directions equivalent to [hk1] by symmetry. In a hexagonal crystal, as found in most silicon carbide polytypes, the Bravais-Miller index scheme is commonly adopted where four indices are used to represent the intercept-reciprocals corresponding to the four principal crystal axes (a1, a2, a3, and c). The axes a1, a2, and a3 are on the same plane and 120° apart from one another while c is perpendicular to the a-plane defined by the (a1, a2, a3) triplet.

Crystalline silicon forms a covalently bonded diamond-cubic structure with lattice constant a=5.43 Å. The diamond-cubic structure is equivalent to two interpenetrating face-centered-cubic (FCC) lattices with basis atoms offset by ¼a in the three orthogonal directions. Silicon's diamond-cubic lattice is relatively sparse (34% packing density) compared to a regular face-centered-cubic (FCC) lattice (74% packing density). Commonly used wafer surface orientations in micromachining include (100), (111), and (110).

Photolithography and etch techniques can create devices in various directions to access desirable material properties. For instance, a (111) oriented piezoresistor in a (110) plane will have the highest piezoresistive sensitivity in a pressure sensor. More commonly, (110) aligned piezoresistors on (100) wafers are used because of their high equal and opposite longitudinal and transverse piezoresistive coefficients.

To define the state of stress for a unit element, nine components, σij, must be specified, as given in the following matrix:

$$σ = [ \begin{matrix}{σ}_{11} & {σ}_{12} & {σ}_{13} \\ {σ}_{21} & {σ}_{22} & {σ}_{23} \\ {σ}_{31} & {σ}_{32} & {σ}_{33}\end{matrix} ] \qquad ( 4 )$$

The first index i denotes the direction of the applied stress, while j indicates the direction of the force or stress. If i=j, the stress is normal to the specified surface, while i≠j indicates a shear stress on face i. From static equilibrium requirements that forces and moments sum to zero, a stress tensor is always symmetric, that is σij=σji, and thus the stress tensor contains only six independent components.

Strain, εij, is also directional. For an isotropic, homogeneous material, stress is related to strain by Hooke's Law, σ=εE. Although “effective” values of Young's modulus and Poisson's ratio for a single direction are often employed for simple loading situations, a tensor is required to fully describe the stiffness of an anisotropic material such as silicon. The stress and strain are related by the elastic stiffness matrix, C, where σij=Cijkl*εkl, or equivalently by the inverse compliance matrix, S, where εij=Sijkl*σkl:

$$[ \begin{matrix}{σ}_{11} \\ {σ}_{22} \\ {σ}_{33} \\ {σ}_{23} \\ {σ}_{13} \\ {σ}_{12}\end{matrix} ] = [ \begin{matrix}{c}_{11} & {c}_{12} & {c}_{13} & {c}_{14} & {c}_{15} & {c}_{16} \\ {c}_{21} & {c}_{22} & {c}_{23} & {c}_{24} & {c}_{25} & {c}_{26} \\ {c}_{13} & {c}_{23} & {c}_{33} & {c}_{34} & {c}_{35} & {c}_{36} \\ {c}_{14} & {c}_{24} & {c}_{34} & {c}_{44} & {c}_{45} & {c}_{46} \\ {c}_{15} & {c}_{25} & {c}_{35} & {c}_{45} & {c}_{55} & {c}_{56} \\ {c}_{16} & {c}_{26} & {c}_{36} & {c}_{46} & {c}_{56} & {c}_{66}\end{matrix} ] [ \begin{matrix}{ε}_{11} \\ {ε}_{22} \\ {ε}_{33} \\ 2 {ε}_{23} \\ 2 {ε}_{13} \\ 2 {ε}_{12}\end{matrix} ] \qquad ( 5 )$$

$$[ \begin{matrix}{ε}_{11} \\ {ε}_{22} \\ {ε}_{33} \\ 2 {ε}_{23} \\ 2 {ε}_{13} \\ 2 {ε}_{12}\end{matrix} ] = [ \begin{matrix}{s}_{11} & {s}_{12} & {s}_{13} & {s}_{14} & {s}_{15} & {s}_{16} \\ {s}_{21} & {s}_{22} & {s}_{23} & {s}_{24} & {s}_{25} & {s}_{26} \\ {s}_{13} & {s}_{23} & {s}_{33} & {s}_{34} & {s}_{35} & {s}_{36} \\ {s}_{14} & {s}_{24} & {s}_{34} & {s}_{44} & {s}_{45} & {s}_{46} \\ {s}_{15} & {s}_{25} & {s}_{35} & {s}_{45} & {s}_{55} & {s}_{56} \\ {s}_{16} & {s}_{26} & {s}_{36} & {s}_{46} & {s}_{56} & {s}_{66}\end{matrix} ] [ \begin{matrix}{σ}_{11} \\ {σ}_{22} \\ {σ}_{33} \\ {σ}_{23} \\ {σ}_{13} \\ {σ}_{12}\end{matrix} ] \qquad ( 6 )$$

Collapsed notation reduces each pair of subscripts to one number: 11→1, 22→2, 33→3, 23→4, 13→5, 12→6, e.g., σ11 to σ1, ε12 to ε6, c1111 to c11 and s2323 to s44.

Single crystal germanium and silicon, both of which have a diamond lattice crystal structure, were the first materials widely used as piezoresistors. Piezoresistive coefficients are used for describing the correlation between the electric field components, the current density and the stress. The piezoresistive coefficients (π) require four subscripts because they relate two second-rank tensors of stress and resistivity. The first subscript refers to the electric field component (measured potential), the second to the current density (current), and the third and fourth to the stress (stress has two directional components). For conciseness, the subscripts of each tensor are also collapsed, e.g., π1111→π11, π1122→π12, π2323→π44. These relations may be generalized for a fixed voltage and current orientation (ω) as a function of stress (λ):

$$\frac{{Δρ}_{ω}}{ρ} = {\sum }_{λ = 1}^{6} {π}_{ωλ} {σ}_{λ} \qquad ( 7 )$$

These coefficients were determined for relatively lightly doped silicon and germanium samples with resistivities ranging from 1.5-22.7 Ω-cm, e.g., 7.8 Ω-cm for p-type silicon. Current commercial and research practice uses doping levels several orders of magnitude higher. Higher concentrations have somewhat lower piezoresistive coefficients, but much lower temperature coefficients of resistance and sensitivity. For example, doping levels are known that result in resistivities in the range of 0.005-0.2 Ω-cm. Piezoresistive coefficients have been measured for (100) samples along the (100) and (110) crystal directions. Longitudinal and transverse coefficients for the fundamental crystal axes were determined directly. Shear piezoresistive coefficients were inferred. By these measurements and considering the crystal symmetry, the piezoresistive tensor of 7.8 Ω-cm silicon was fully characterized according to Smith, C. S. (1954) Piezoresistance Effect in Germanium and Silicon. Physical Review, 94, 42-49, as follows:

$$[ {π}_{ωλ} ] = [ \begin{matrix}{π}_{11} & {π}_{12} & {π}_{12} & 0 & 0 & 0 \\ {π}_{12} & {π}_{22} & {π}_{12} & 0 & 0 & 0 \\ {π}_{12} & {π}_{12} & {π}_{33} & 0 & 0 & 0 \\ 0 & 0 & 0 & {π}_{44} & 0 & 0 \\ 0 & 0 & 0 & 0 & {π}_{44} & 0 \\ 0 & 0 & 0 & 0 & 0 & {π}_{44}\end{matrix} ] = \qquad ( 8 )$$

$$[ \begin{matrix}6.6 & - 1.1 & - 1.1 & 0 & 0 & 0 \\ - 1.1 & 6.6 & - 1.1 & 0 & 0 & 0 \\ - 1.1 & - 1.1 & 6.6 & 0 & 0 & 0 \\ 0 & 0 & 0 & 138.1 & 0 & 0 \\ 0 & 0 & 0 & 0 & 138.1 & 0 \\ 0 & 0 & 0 & 0 & 0 & 138.1\end{matrix} ] \times {10}^{- 11} {Pa}^{- 1}$$

For a p-type piezoresistor implanted on (100) silicon along the (100) crystal direction, the transversal and longitudinal piezoresistive coefficients may be defined and approximated as follows:

$${π}_{t} = \frac{1}{2} ( {π}_{11} + {π}_{12} - {π}_{44} ) = - \frac{{π}_{44}}{2} \qquad ( 9 )$$

$${π}_{l} = \frac{1}{2} ( {π}_{11} + {π}_{12} + {π}_{44} ) = \frac{{π}_{44}}{2} \qquad ( 10 )$$

Thus, the resulting change in resistance is

$$\frac{Δ R}{R} = {σ}_{l} {π}_{l} + {σ}_{t} {π}_{t} \qquad ( 11 )$$

$$\frac{Δ R}{R} = {π}_{44} ( {σ}_{l} - {σ}_{t} ) \qquad ( 12 )$$

FIG. 10 illustrates a simulation of the stress values σ₁-σₜ for a membrane 202 as depicted in FIG. 9 when a pressure of 1 bar is applied from the back. In this example, the membrane has dimensions of 390 μm in the x direction, 390 μm in the y direction, and a thickness of 6 μm.

It should be noted that a difference has to be made between the terms stress and strain.

When a material is put under pressure or has a mechanical load applied to it, mechanical stress is developed. When a solid is put under stress, it has the ability to deform. This deformation is called strain. The stress is the pressure per unit area of the material, and the resulting strain is the deformation that occurs as a result of this stress. Strain and stress are strongly intertwined because strain occurs solely as a result of stress. Stress is defined as the force per unit area generated within materials as a result of externally applied forces, unequal heating, or persistent deformation. The unit for stress is Nm-2 (Pa). On the other hand, strain is defined as the amount of distortion experienced by the body in the direction of force compared to the original dimension of the object. Strain defines the relative change in the shape of an object.

The piezoresistors used for the piezoresistive sensor elements according to the present disclosure are responsive to the stress they experience.

Furthermore, it is known in the art to use the concept of a Wheatstone bridge in order to ensure a precise and offset free measurement. FIG. 11 illustrates a schematic representation of a MEMS pressure sensor 200 comprising four stress sensitive piezoresistors R1, R2, R3, R4. A deflectable membrane (also referred to as diaphragm) 202 is surrounded by a stiffer frame 204. Pressure that acts on the membrane 202 causes stress in the membrane which is sensed by the four stress sensitive piezoresistors R1, R2, R3, R4.

These piezoresistors are arranged in the Wheatstone bridge circuit as depicted in FIG. 12 for a full-bridge configuration. When no pressure is applied, the output voltage, Vout is 0 V as all the piezoresistors are equal to R₀ (1+αΔT). When the pressure is applied, the diaphragm is under stress. The active piezoresistors, which are positioned in the high stress regions on the diaphragm are strained, hence change their values to R₀ (1+αΔT+π₁ Δσ₁+πₜ Δσₜ) where a is the temperature coefficient of resistance. π₁ and πₜ are the longitudinal and transverse piezoresistive coefficients, respectively. Meanwhile ΔT, Δσ₁, and Δσₜ are the changes in temperature, longitudinal stress and transverse stress, respectively. For example with pressure coming from the top of the membrane, the active perpendicular resistors (R1 and R3) experience mostly the longitudinal stress and the active parallel resistors (R2 and R4) undergo mostly the transverse stress. In other words, the resistors all experience both stresses. The sum of these two stresses is drawn in FIG. 10, illustrating why two of these resistors increase in value while the two others decrease when the membrane is exposed to pressure. A longitudinal tensile stress increases the resistivity of p-type resistors, while a transverse stress has the opposite effect. Hence, the Vout changes accordingly due to these changes. Assume that ΔR1=ΔR3 and ΔR2=ΔR4, thus, the Vout expressions for the full-bridge (FIG. 13) are given by the following expression:

$$Vout = Vin ( \frac{{π}_{l} Δ {σ}_{l} - {π}_{t} {Δσ}_{t}}{2 ( 1 + αΔ T ) + {π}_{l} {σ}_{l} - {π}_{t} {Δσ}_{t}} ) \qquad ( 13 )$$

As this is generally known, many disturbances that act uniformly on all four resistors can be eliminated by using the Wheatstone bridge configuration. Examples of such disturbances include process variations such as an implant dose, a linewidth and changes of the resistors due to temperature.

However, it has been found that conventional piezoresistive sensors suffer from slow long-term drift effects that are for instance caused by electrostatic build-up (for instance in a covering gel or a plastic cap) and/or environmental effects and/or process issues such as ionic contamination, material impurities, electromigration, stress release, stress in material layers, process residual stress, thermal coefficient (TC) mismatch, TC change etc. Stress isolation may cause such a drift, but this effect is not considered for the present disclosure because there exist concepts to limit this drift effect.

A slow drift of the offset caused by external disturbances cannot be discerned from slow changes of the measurand. In particular for the application of a MEMS pressure sensor, this drawback leads to a functional safety concern. In particular, relevant standards in the application fields of automotive industry, or safety in medical applications such as for respiratory devices In the context of the present disclosure, the term long-term drift relates to time spans between several months and about two years, preferably about one year.

Some conventional sensor concepts deal with this problem using a buried field shield for compensating the effect of any ionic contaminations due to the fabrication process. Further, it is known to use a polycrystalline silicon or metal field shield to nullify external electric fields. Some conventional sensors comprise a sensor housing having metal caps with lid shields in order to avoid electrostatic charge build-up. However, these counter-measures complicate the manufacturing process and often do not achieve satisfactory results.

Moreover, EP 3287758 B1 discloses a differential pressure sensor, which may provide a common mode corrected differential pressure reading. The differential pressure sensor includes for instance two pressure sensing diaphragms. The pressure sensor may be configured so that the first diaphragm measures the differential pressure between two sections of a fluid. The pressure sensor may also be configured so that the second diaphragm measures the common mode error experienced by the die at the time the differential pressure is read by the first diaphragm. Electrical connectors may be configured so that the differential pressure outputs a common mode error corrected differential pressure reading based on the readings of the first and second diaphragm. In particular, the second diaphragm includes at least one pressure sensitive electrical element that exhibits a varying resistance responsive to deflection of the diaphragm of the second pressure sensing die that is representative of a common mode error of the second pressure sensing die.

European patent application 22198016.2 relates to another differential pressure sensor. According to this document, the drift signal of the drift pressure sensor (i.e. a signal generated by a drift sensing unit formed on or in the symmetrical diaphragm) can additionally be used to trigger a warning signal. In more detail, by comparing the drift signal with a predefined threshold value, it is possible to estimate whether the differential pressure sensor (in particular a signal generated by a differential sensing unit formed on or the differential diaphragm) has been degraded.

This enables the technical effect that the differential pressure sensor can have a longer lifetime, in particular the pressure sensor does not need to be replaced after a predefined fixed time but can be replaced on demand (namely when the warning signal, which is sensed by the drift pressure sensor, is output).

However, there is still the need for a piezoresistive sensor, which allows a particularly precise and long-term stable measurement, at the same time being fabricated in a cost efficient manner.

### BRIEF DESCRIPTION OF THE INVENTION

Various embodiments disclosed herein have an object of a piezoresistive sensor, which allows a particularly precise and long-term stable measurement, at the same time being fabricated in a cost efficient manner. Advantageous embodiments of the present disclosure are the subject matter of the claims.

The present disclosure is based on the idea to provide a second Wheatstone bridge in addition to the main bridge for monitoring and compensating environmental and/or process induced drift effects. In particular, a piezoresistive sensor element according to the present disclosure comprises a substrate, which in operation is subjected to mechanical stress in response to a measurand to be measured, and a first array of at least four sensitive piezoresistors, wherein the sensitive piezoresistors are arranged on the substrate and are connected to form a first Wheatstone bridge for generating a first bridge signal. Further, a second array of at least four insensitive piezoresistors is provided, wherein the insensitive piezoresistors are connected to form a second Wheatstone bridge for generating a second bridge signal, and wherein the sensitive piezoresistors have a stress sensitivity which is higher than the stress sensitivity of the insensitive piezoresistors, and wherein an output signal of the piezoresistive sensor is generated based on a difference of the first and second bridge signals.

This solution has the advantage that by subtracting the output signals of the two bridges long-term drift can be compensated, while the sensitivity of the main bridge remains unaffected. Thus, the reliability and safety of the sensor is enhanced.

According to an advantageous example of the piezoresistive sensor element, the substrate comprises silicon, wherein each of the piezoresistors comprises doped areas ion-implanted in the silicon material. Silicon is a well-established material in the semiconductor industry and offers the potential for using established CMOS processes for fabricating the sensor element and further electronic components. As mentioned in the theoretical part above, monocrystalline silicon also exhibits a strong piezoresistive effect, in particular when using ion implantation for fabricating the piezoresistors.

However, it is clear that other materials showing a piezoresistive effect (for instance germanium or silicon carbide) may also be used in connection with the present disclosure. Further, for the fabrication of the piezoresistors, also other known technologies such as diffusion, epitaxy, or deposition of a doped polycrystalline silicon layer can also be employed.

When manufacturing the piezoresistive sensor element using a monocrystalline material, such as monocrystalline silicon, the piezoresistivity may be anisotropic. Advantageously, each of the sensitive piezoresistors may be oriented along a first crystallographic orientation of the substrate and each of the corresponding insensitive piezoresistors may be oriented along a second crystallographic orientation of the substrate, the first crystallographic orientation being different from the second crystallographic orientation and causing a higher stress sensitivity than the second crystallographic orientation.

For instance, the substrate may be fabricated from p-type silicon with a (100) plane forming the outer surface, wherein the sensitive piezoresistors are arranged along a [1 10] direction and/or a [1 10] direction, and wherein the insensitive piezoresistors are arranged along a [1 00] direction and/or a direction. A particularly high sensitivity and accuracy can be achieved with this configuration.

Alternatively, the substrate may also be fabricated from n-type silicon with a (100) plane forming the outer surface, wherein the sensitive piezoresistors are arranged along a [1 00] direction and/or a [01 0] direction and/or a direction, and wherein the insensitive piezoresistors are arranged along a direction and/or a [1 10] direction.

In order to ensure that the elements of the second Wheatstone bridge experience the same disturbances as the piezoresistors of the first Wheatstone bridge, so that all long-term drift effects match as closely as possible, each of the insensitive piezoresistors may be arranged in close proximity to one corresponding sensitive piezoresistor, so as to be subjected to essentially the same stress as the corresponding sensitive piezoresistor.

According to an advantageous example of the piezoresistive sensor element according to the present disclosure, the insensitive piezoresistors are arranged to include an angle of 45 degrees with each of the sensitive piezoresistors.

According to a further advantageous example, the piezoresistive sensor element further comprises a signal processing unit for evaluating the first bridge signal and the second bridge signal and for generating the sensor output signal. Thus, no external signal processing units and connection leads have to be involved for the compensation calculus, so that the accuracy and reliability can be increased.

The most accurate and compact architecture can be achieved when the signal processing unit is monolithically integrated with the piezoresistors on the same chip (also called die).

The effect of temperature influences may be monitored either be using the inherent temperature coefficient of resistance (TCR) of the second Wheatstone bridge or by additionally providing a temperature sensor, such as a temperature diode, arranged within the second Wheatstone bridge. Such a temperature diode is also known as a thermal diode. The functioning of a thermal diode is based on the property of electrical diodes to change voltage across it linearly according to temperature.

As mentioned above, the architecture proposed in the present disclosure is intended to eliminate the detrimental effects of long-term drift effects due to electrostatic build-up or external or process induced ionic influences. In order to enhance the effect of these influences on the second Wheatstone bridge, the passivation layer, which is present on the piezoresistive sensor element, may at least partly removed in regions above the insensitive piezoresistors.

The present disclosure relates exemplarily to a piezoresistive pressure sensor comprising at least one piezoresistive sensor element according to the principles described above, wherein the substrate comprises a deflectable membrane (which may also be referred to as a diaphragm), which in operation is deflected in response to a pressure to be measured.

It is clear that the ideas of the present disclosure may also be applicable to other piezoresistive sensors, such as force and inertial sensors. For instance, cantilever sensors, strain gauges, accelerometers, and gyroscopes may be equipped with piezoresistive sensor elements in line with the present disclosure. However, piezoresistive pressure sensors are some of the most reported and developed micromachined devices. Thus, the following detailed description will focus on piezoresistive pressure sensors, which typically measure deformation of a thin circular or rectangular membrane (diaphragm) under an applied external pressure. The membrane may be made from the same material as the wafer substrate (silicon, diamond, etc.) or CVD-based thin films (oxide, nitride, etc.). Integrated piezoresistors are formed by dopant diffusion, ion implantation, or doped epitaxy. However, it is clear that other materials showing a piezoresistive effect (for instance germanium or silicon carbide) may also be used in connection with the present disclosure.

According to an advantageous example, the membrane is surrounded by a frame having a higher stiffness than the membrane, and wherein the insensitive piezoresistors are arranged on the frame. Thus, the in-insensitive piezoresistors are decoupled from deformation of the membrane. The insensitive piezoresistors may in this case be arranged close to the periphery of the diaphragm or further away from the diaphragm. However, the insensitive piezoresistors are arranged on the deflectable membrane itself, so as to experience exactly the same stress and environment as the sensitive piezoresistors.

According to an advantageous example, the membrane has a rectangular outline and the sensitive piezoresistors have an elongated shape, and wherein a first pair of the sensitive piezoresistors are arranged along opposing sides of the deflectable membrane's outline and a second pair of the sensitive piezoresistors are arranged to include an angle with the other opposing sides of the deflectable membrane's outline.

The accompanying drawings are incorporated into the specification and form a part of the specification to illustrate several embodiments of the present disclosure. These drawings, together with the description serve to explain the principles of the disclosure. The drawings are merely for the purpose of illustrating the preferred and alternative examples of how the disclosure can be made and used, and are not to be construed as limiting the disclosure to only the illustrated and described embodiments. Furthermore, several aspects of the embodiments may form-individually or in different combinations-solutions according to the present disclosure. The following described embodiments thus can be considered either alone or in an arbitrary combination thereof.

### BRIEF DESCRIPTION OF THE DRAWINGS

Further features and advantages will become apparent from the following more particular description of the various embodiments of the disclosure, as illustrated in the accompanying drawings, in which like references refer to like elements, and wherein:

FIG. 1 is a schematic top view of a piezoresistive pressure sensor according to a first example;

FIG. 2 is a schematic is a schematic cross sectional view of the piezoresistive pressure sensor shown in FIG. 1;

FIG. 3 is a schematic representation illustrating the crystallographic orientation of the piezoresistors according to an advantageous example;

FIG. 4 is a schematic representation of the electronic signal processing according to an advantageous example;

FIG. 5 is a schematic top view of a piezoresistive pressure sensor according to a second example;

FIG. 6 is a schematic is a schematic cross sectional view of a piezoresistive pressure sensor according to a further example;

FIG. 7 is a schematic top view of a piezoresistive pressure sensor according to a further example;

FIG. 8 is a schematic representation of the electronic signal processing according to a further advantageous example;

FIG. 9 is a schematic top view of a diaphragm region of a conventional piezoresistive pressure sensor;

FIG. 10 is schematic illustration of the stress distribution within the piezoresistive pressure sensor of FIG. 9;

FIG. 11 is a schematic top view of a conventional piezoresistive pressure sensor;

FIG. 12 is a circuit diagram of a Wheatstone bridge comprising the piezoresistors shown in FIG. 11.

### DETAILED DESCRIPTION OF THE INVENTION

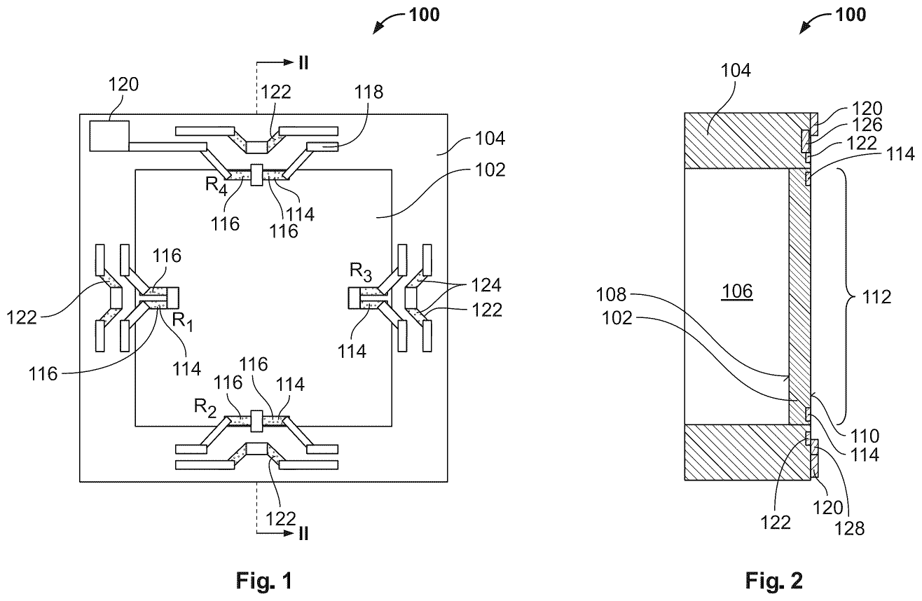

The present disclosure will now be explained in more detail with reference to the Figures and firstly referring to FIGS. 1 and 2.

FIG. 1 is a schematic top view of a piezoresistive pressure sensor 100 according to a first example of the present disclosure. FIG. 2 is the corresponding sectional view along the cut line II-II of FIG. 1.

According to this particular example, the piezoresistive pressure sensor 100 has a rectangular outline. Of course, the principles according to the present disclosure may also be applied to sensor structures with a different outline, e.g. a circular outline. The piezoresistive pressure sensor 100 comprises a deflectable membrane 102 which is also referred to as a diaphragm. The membrane 102 is surrounded and supported by a thicker and thus stiffer frame 104.

The membrane 102 and the frame 104 define a void 106, in which the pressure to be measured is allowed to act on the first (inner) side 108 of the membrane. The second side 110 of the membrane is oriented outwardly.

When a pressure difference is built up between the first side 108 and the second side 110 of the membrane 102, mechanical stress is generated. The membrane is deflectable and the maximal stress occurs on the edge. This may for instance be seen from FIG. 10. The maximum stress is therefore generated in the area 112 defined by the membrane 102. The frame 104 is the location where the stress caused by the pressure is minimal.

According to the present disclosure, a group of four sensitive piezoresistors 114 is arranged on the membrane 102 around the perimeter of the membrane 102. According to the shown example, each of the sensitive piezoresistors 114 comprises one or more, for instance two, ion implanted resistive regions 116 and electrically conductive connecting leads 118, which interconnect the resistive regions 116 and allow the interconnection between the sensitive piezoresistors 114 and to a signal processing circuit. Bond pads 120 may be provided for the electrical connection of the die for instance by wire bonding, solder bumps in flip-chip technology, or the like. In FIGS. 1 and 2, only one of the pads 120 is shown in order to keep the schematic drawing simple.

The sensitive piezoresistors 114 in operation of the pressure sensor 100 change their resistivity in response to mechanical stress in the membrane with the maximum possible sensitivity. The four piezoresistors 114 are interconnected with each other to form a Wheatstone bridge circuit as explained above and shown in FIG. 12.

According to the present disclosure, in addition to these sensitive piezoresistors 114, a group of insensitive piezoresistors 122 is provided for compensating the effects of long-term drift. The insensitive piezoresistors 122 react towards mechanical stress with no or only a very low sensitivity. Thus, these insensitive piezoresistors 122 are essentially only perceptive of the drift causing influences. The insensitive piezoresistors 122 are connected to form a second Wheatstone bridge and the output signal of the second Wheatstone bridge can be subtracted from the output signal of the first Wheatstone bridge in order to yield an accurate and drift-compensated sensor signal.

The insensitive piezoresistors 122 may each comprise one or more interconnected ion implanted resistive regions 124.

As schematically illustrated in FIG. 2, the connecting leads 118 may for instance comprise diffused conductors 126 or deposited conductive layers, e.g. metal layers 128.

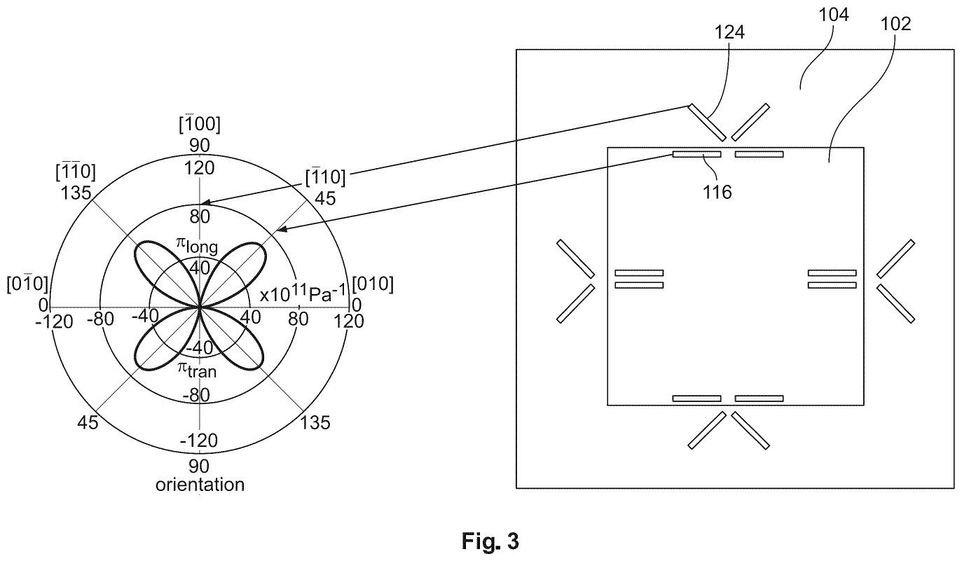

The example shown in FIGS. 1 and 2 shows a piezoresistive pressure sensor 100, which is based on p-type monocrystalline silicon. As can be derived from FIG. 1, the resistive regions 124 of the stress insensitive piezoresistors 122 are laid out at an angle of 45 degrees compared to the resistive regions 116 of the stress sensitive piezoresistors 114. The reason for this arrangement will now be explained with reference to FIG. 3.

In particular, in FIG. 3 the orientation of the ion implanted resistive regions 116 of the stress sensitive piezoresistors 114 and the ion implanted resistive regions 124 of the stress insensitive piezoresistors 122 is compared to the piezoresistive coefficients in the (100) plane of a p-type silicon substrate as for instance shown in Barlian, A. Alvin & Park, Woo-Tae & Mallon, Joseph R., Jr. & Rastegar, Ali J. & Pruitt, Beth L. (2009): “Review: Semiconductor Piezoresistance for Microsystems”, Proceedings of the IEEE. Institute of Electrical and Electronics Engineers, 97, pp. 513-552, DOI: 10.1109/JPROC.2009.2013612.

The ion implanted resistive regions 116 of the stress sensitive piezoresistors 114 are arranged along the [1 1 0] direction and the [1 10] direction, so that these piezoresistors exhibit the maximum possible stress sensitivity. In contrast thereto, the ion implanted resistive regions 124 of the stress insensitive piezoresistors 122 are arranged along the [1 00] direction and the direction, thus exhibiting no or only a negligible stress sensitivity.

Although not shown in the Figures, it should be noted that for n-type silicon the sensitive piezoresistors have to be arranged along the [1 00] direction, the [01 0] direction and/or the direction, whereas the insensitive piezoresistors are arranged along the [1 1 0] direction and/or the [1 10] direction, in order to achieve an analogous result.

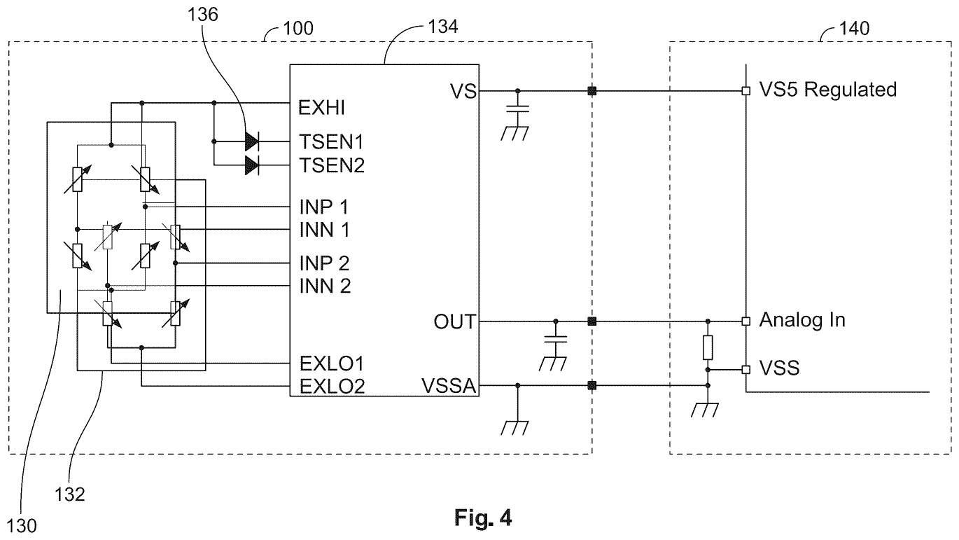

FIG. 4 illustrates an example of a sensor element 100, which integrally comprises the first Wheatstone bridge 130 with the sensitive piezoresistors, the second Wheatstone bridge 132 with the insensitive piezoresistors, and an integrated signal processing unit 134. The integrated signal processing unit 134 is operable to calculate a drift-compensated sensor output signal. Two temperature sensors (in particular, temperature diodes) 136 are provided for sensing the temperature of the sensor element 100, so that further compensation calculations can be performed, thus rendering the output signal even more accurate.

All components may for instance be integrated as one ASIC together with an electronic control circuit (ECU) 140.

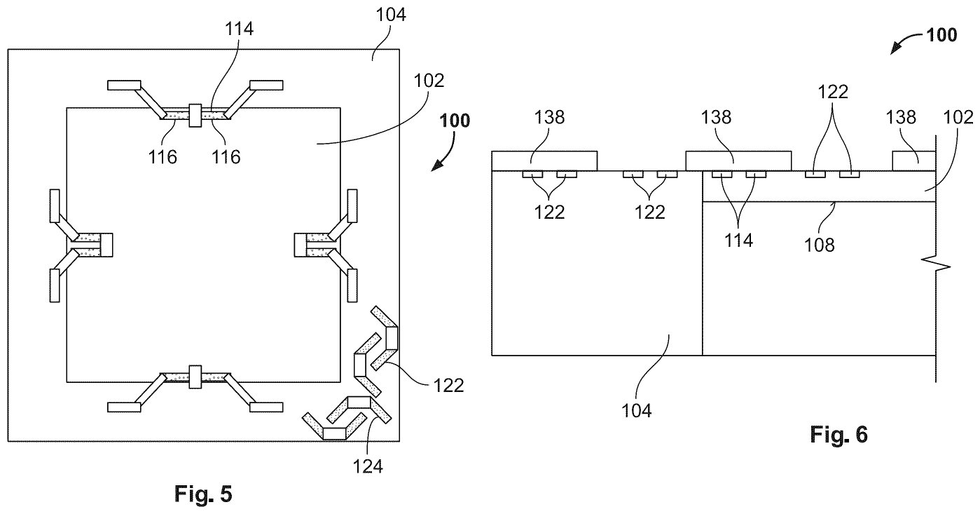

According to the example shown in FIGS. 1 and 2, each of the insensitive piezoresistors 122 is placed in close vicinity to a corresponding sensitive piezoresistor 114. However, this does not necessarily have to be the case. The insensitive piezoresistors 122 may alternatively be located distanced away from the sensitive piezoresistors 114. An example of such an arrangement is depicted in FIG. 5. Here, the insensitive piezoresistors 122 are arranged in a peripheral region of the frame 104. Again, the resistive regions 124 of the stress insensitive piezoresistors 122 are laid out at an angle of 45 degrees compared to the resistive regions 116 of the stress sensitive piezoresistors 114. Thus, the stress sensitivity of insensitive piezoresistors 122 is zero or negligible compared to the stress sensitivity of the sensitive piezoresistors 114.

It has been found that the long-term drift effects that are to be tackled according to the present disclosure are partly screened by the usually applied passivation layers. The passivation for instance comprises a stack of a layer of silicon dioxide followed by a layer of silicon nitride. This finding is taken into account for the exemplary arrangement shown in FIG. 6.

According to FIG. 6, the passivation layer 138 is partly removed to augment the impact of the drift. In particular, the passivation layer 138 is removed from the regions where the insensitive piezoresistors 122 are located to facilitate the effects of the drift.

Furthermore, the backside 108 of the membrane 102 can also be a cause of drift due to electrical charges or due to leakage current, if the depth of the piezoresistors is close to the thickness of the membrane 102. As shown in FIG. 6, the insensitive piezoresistors 122 can be arranged on the frame 104 to make them as insensitive to stress as possible or on the membrane 102 to imitate the situation of the stress sensitive piezoresistors 114.

In FIG. 6, reference numeral 122 indicates stress insensitive resistors, whereas reference numeral 114 designates the stress sensitive piezoresistors. Another possibility would be to have the resistors 122 with a passivation on the membrane (i.e. the same arrangement as for the resistors 114, but in a stress insensitive orientation), but that arrangement would be less advantageous.

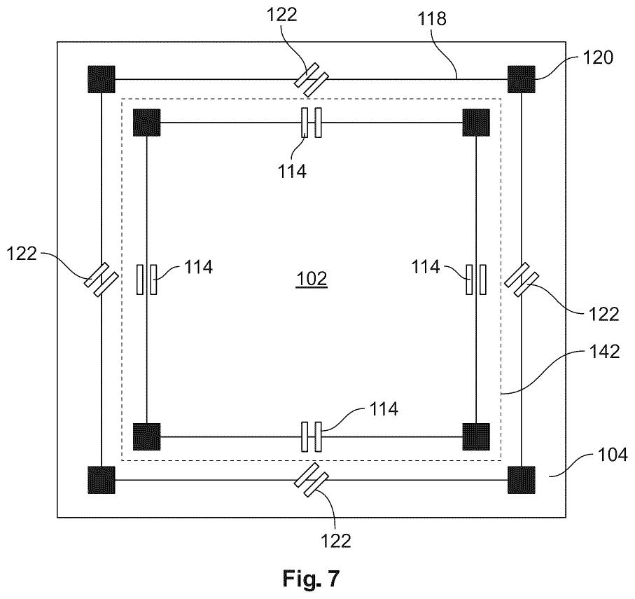

FIG. 7 illustrates still another advantageous example of a piezoresistive pressure sensor 100. The periphery of the membrane is marked here by a broken line 142. Again, the resistive regions of the stress insensitive piezoresistors 122 are laid out at an angle of 45 degrees compared to the resistive regions of the stress sensitive piezoresistors 114. Thus, the stress sensitivity of insensitive piezoresistors 122 is zero or negligible compared to the stress sensitivity of the sensitive piezoresistors 114.

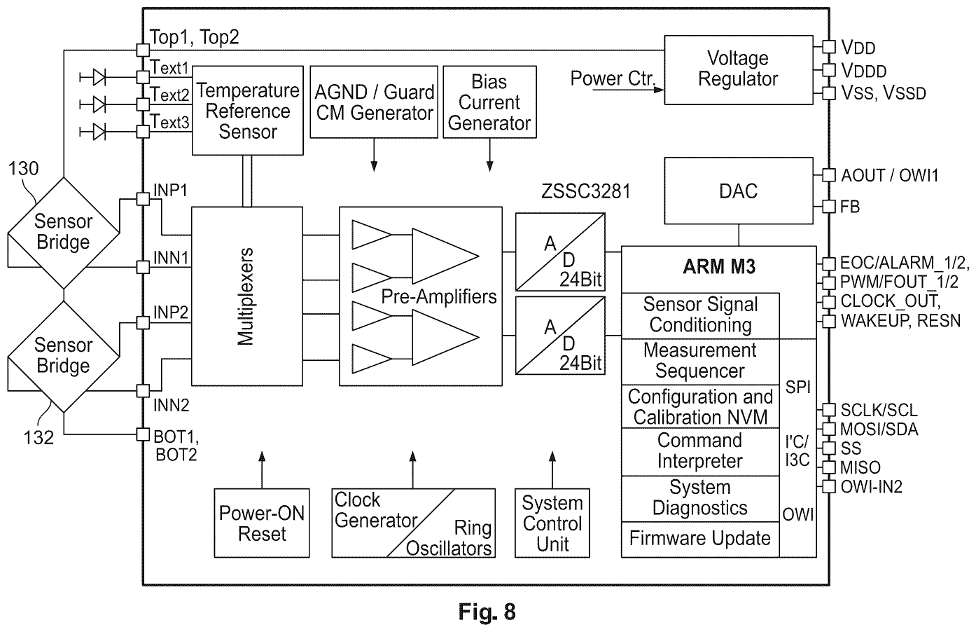

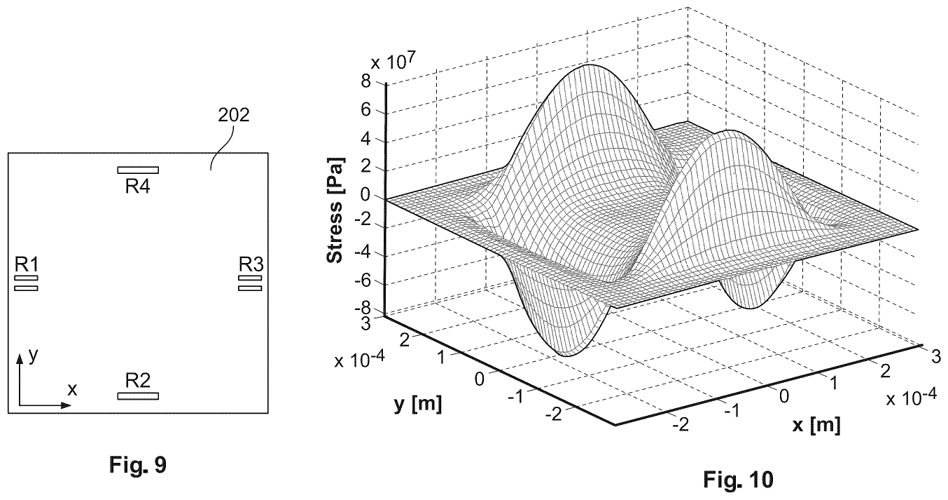

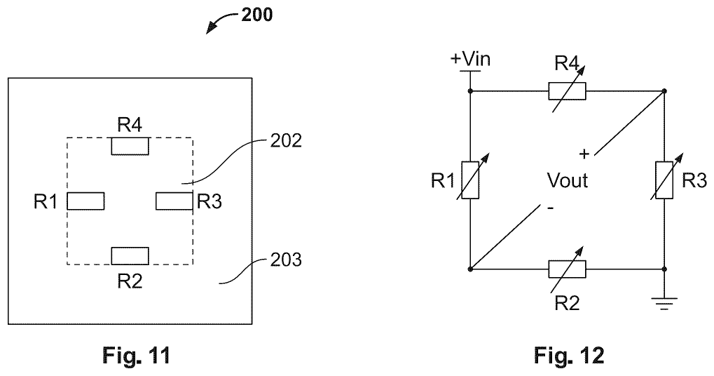

FIG. 8 illustrates a further example of an electronic circuit that can used for calculating the compensated sensor output signal from the output signals of the first Wheatstone bridge 130 with the sensitive piezoresistors and the second Wheatstone bridge 132 with the insensitive piezoresistors.

A compensation using an ASIC such as the dual channel 24-Bit resistive sensor signal conditioner with analog and digital output ZSSC3281 sold by the company Renesas Electronics is a mathematical operation using the output of the stress sensitive bridge and stress insensitive bridge. An example is a subtraction of the stress insensitive output (i.e. the drift) from the stress sensitive output. Another example is to use the output of the stress insensitive bridge to trigger a warning when it exceeds a certain amount of drift.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112 (f), unless and until such claim limitations expressly use the phrase “means for” followed by a statement of function void of further structure.

What is claimed is:

1. Piezoresistive sensor element comprising:
a substrate, which in operation is subjected to mechanical stress in response to a measurand to be measured,
a first array of at least four sensitive piezoresistors, wherein the sensitive piezoresistors are arranged on the substrate and are connected to form a first Wheatstone bridge for generating a first bridge signal,
a second array of at least four insensitive piezoresistors, wherein the insensitive piezoresistors are connected to form a second Wheatstone bridge for generating a second bridge signal,
wherein the sensitive piezoresistors have a stress sensitivity which is higher than a stress sensitivity of the insensitive piezoresistors, and wherein an output signal of the piezoresistive sensor is generated based on a difference of the first and second bridge signals.

2. Piezoresistive sensor element according to claim 1, wherein the substrate comprises silicon, and wherein each of the piezoresistors comprises doped areas ion-implanted in the silicon material.

3. Piezoresistive sensor element according to claim 1, wherein each of the sensitive piezoresistors is oriented along a first crystallographic orientation of the substrate and each of the corresponding insensitive piezoresistors is oriented along a second crystallographic orientation of the substrate, the first crystallographic orientation being different from the second crystallographic orientation and causing a higher stress sensitivity than the second crystallographic orientation.

4. Piezoresistive sensor element according to claim 1, wherein the substrate is fabricated from p-type silicon with a (100) plane forming the outer surface, and wherein the sensitive piezoresistors are arranged along a [1 1 0] direction and/or a [1 10] direction, and wherein the insensitive piezoresistors are arranged along a [1 00] direction and/or a direction.

5. Piezoresistive sensor element according to claim 1, wherein the substrate is fabricated from n-type silicon with a (100) plane forming the outer surface, and wherein the sensitive piezoresistors are arranged along a [1 00] direction and/or a [01 0] direction and/or a direction, and wherein the insensitive piezoresistors are arranged along a [1 1 0] direction and/or a [1 10] direction.

6. Piezoresistive sensor element according to claim 1, wherein each of the insensitive piezoresistors is arranged in close proximity to one corresponding sensitive piezoresistor, so as to be subjected to essentially the same stress as the corresponding sensitive piezoresistor.

7. Piezoresistive sensor element according to claim 1, wherein the insensitive piezoresistors are arranged to include an angle of 45 degrees with each of the sensitive piezoresistors.

8. Piezoresistive sensor element according to claim 1, further comprising a signal processing unit for evaluating the first bridge signal and the second bridge signal and for generating the sensor output signal.

9. Piezoresistive sensor element according to claim 8, wherein the signal processing unit is monolithically integrated with the piezoresistors.

10. Piezoresistive sensor element according to claim 1, further comprising a temperature sensor arranged within the second Wheatstone bridge.

11. Piezoresistive sensor element according to claim 1, further comprising at least one passivation layer, wherein the passivation layer is at least partly removed in regions above the insensitive piezoresistors.

12. Piezoresistive pressure sensor comprising:
a piezoresistive sensor element including a substrate, a first array of at least four sensitive piezoresistors, and a second array of at least four insensitive piezoresistors,
wherein the substrate comprises a deflectable membrane configured to be deflected in response to a pressure to be measured, the substrate being subjected to mechanical stress in response to a measurand to be measured,
wherein the sensitive piezoresistors are arranged on the substrate and are connected to form a first Wheatstone bridge for generating a first bridge signal,
wherein the insensitive piezoresistors are connected to form a second Wheatstone bridge for generating a second bridge signal,
wherein the sensitive piezoresistors have a stress sensitivity which is higher than a stress sensitivity of the insensitive piezoresistors, and wherein an output signal of the piezoresistive sensor is generated based on a difference of the first and second bridge signals.

13. Piezoresistive pressure sensor according to claim 12, wherein the membrane is surrounded by a frame having a higher stiffness than the membrane, and wherein the insensitive piezoresistors are arranged on the frame.

14. Piezoresistive pressure sensor according to claim 12, wherein the membrane is surrounded by a frame having a higher stiffness than the membrane, and wherein the insensitive piezoresistors are arranged on the deflectable membrane.

15. Piezoresistive pressure sensor according to claim 12, wherein the membrane has a rectangular outline and the sensitive piezoresistors have an elongated shape, and wherein a first pair of the sensitive piezoresistors are arranged along opposing sides of the deflectable membrane's outline and a second pair of the sensitive piezoresistors are arranged to include an angle with the other opposing sides of the deflectable membrane's outline.

16. Piezoresistive pressure sensor according to claim 12, wherein the substrate comprises silicon, and wherein each of the piezoresistors comprises doped areas ion-implanted in the silicon material.

17. Piezoresistive pressure sensor according to claim 12, wherein each of the sensitive piezoresistors is oriented along a first crystallographic orientation of the substrate and each of the corresponding insensitive piezoresistors is oriented along a second crystallographic orientation of the substrate, the first crystallographic orientation being different from the second crystallographic orientation and causing a higher stress sensitivity than the second crystallographic orientation.

18. Piezoresistive pressure sensor according to claim 12, wherein the substrate is fabricated from p-type silicon with a (100) plane forming the outer surface, and wherein the sensitive piezoresistors are arranged along a [1 1 0] direction and/or a [1 10] direction, and wherein the insensitive piezoresistors are arranged along a [1 00] direction and/or a direction.

19. Piezoresistive pressure sensor according to claim 12, wherein the substrate is fabricated from n-type silicon with a (100) plane forming the outer surface, and wherein the sensitive piezoresistors are arranged along a [1 00] direction and/or a [01 0] direction and/or a direction, and wherein the insensitive piezoresistors are arranged along a [1 1 0] direction and/or a [1 10] direction.

20. Piezoresistive pressure sensor according to claim 12, wherein each of the insensitive piezoresistors is arranged in close proximity to one corresponding sensitive piezoresistor, so as to be subjected to essentially the same stress as the corresponding sensitive piezoresistor.
